(19) United States
(12) Patent Application Publication
(10) Pub. No.: US 2025/0288375 A1
(43) Pub. Date: Sep. 18, 2025

(54) 3D PINLESS NAVIGATION/ROBOTIC SYSTEM USING TEMPORARY SUTURE ANCHORS

(71) Applicant: Jesua Law, Ripon, CA (US)

(72) Inventor: Jesua Law, Ripon, CA (US)

(21) Appl. No.: 19/076,742

(22) Filed: Mar. 11, 2025

Related U.S. Application Data
(60) Provisional application No. 63/566,250, filed on Mar. 16, 2024.

(51) Int. Cl. A61B 34/30; A61B 17/04; A61B 34/20; A61B 90/00

(52) U.S. Cl. CPC A61B 34/30; A61B 17/0401; A61B 90/39; A61B 2034/2072; A61B 2090/3916; A61B 2090/3991

(57) ABSTRACT

A navigation system is used to provide the real-time position of a patient's anatomy during the procedure. An innovative suture anchor is used to temporarily affix a navigation marker to a patient's anatomy for an orthopedic procedure. The suture anchor includes a first suture having a hollow passageway connecting a knot end and a leash end, a second suture having a hollow passageway connecting a knot end and a leash end, and a third suture having two leash ends and passing through the hollow passageways of the first and second sutures and interconnecting the knot ends thereof. When the two leash ends of the third suture is pulled it causes the knot ends of the first and second sutures to interlock and expand into an enlarged knot, and the enlarge knot is adapted to engage a small diameter superficial hole in a patient's bone.

### RELATED APPLICATION

The present application claims the benefit of U.S. Provisional Patent Application 63/566,250 filed on Mar. 16, 2024, the entirety of which is incorporated herein by reference.

### FIELD

The present disclosure relates to systems and methods for minimally invasive robotic or navigated orthopedic surgery using temporary suture anchors to affix navigational markers to the patient's anatomy.

### BACKGROUND

Joint replacement surgeries, including knee, hip, shoulder or ankle replacement procedures, are commonly used to replace a patient's joint with a prosthetic joint component. Performing such procedures when using robotics or navigation will require the temporary insertion of reference pins into the bones of patients at specific spots for measuring the complex spatial anatomy relative to these reference points. In addition, instrumentation into the complex human spinal anatomy usually is performed with the assistance of navigation and traditional reference pins. The traditional reference pins are of large diameter and are placed by drilling into the patient's bones. These traditional reference pins may loosen from the bone during the procedure, leading to measurement errors. These reference pins must stay stationary throughout the entire surgery and thus may obstruct the surgeon during portions of the procedure. The presence and subsequent removal of these pins can alter the way stress is distributed across the patient's bone(s), potentially leading to areas of increased stress. The holes left behind in the patient's bones also may create areas of weakness in the bone, potentially increasing the risk of fractures. Post-surgery the patient also has the burden of healing from the drilled pin holes, which has been shown to be a source of ongoing pain even after complete healing from the navigated/robotic orthopedic surgery.

### BRIEF DESCRIPTION OF THE DRAWINGS

A better understanding of the claimed subject matter can be obtained when the following detailed description of the disclosed embodiments is considered in conjunction with the following figures.

FIG. 1 illustrates how a computerized system is used to determine and map the position and orientation of a patient's pelvis.

FIG. 2 is a simplified block diagram of the computerized system that enable robotic guided surgical procedures according to the teachings of the present disclosure.

FIGS. 3A-3C depict the pelvis and hip joint further defining the pelvic planes and the hip coordinate system.

FIG. 4 is a cross-sectional view of a first embodiment of a temporary suture anchor that may be used to temporarily secure a reference marker or tracker to a patient's anatomy through a small cortical hole according to the teachings of the present disclosure.

FIG. 5 is a cross-sectional view of a second embodiment of a temporary suture anchor that may be used to temporarily secure a reference marker or tracker to a patient's anatomy through a small cortical hole using a beanbag and a supporting platform according to the teachings of the present disclosure.

FIGS. 6-12 illustrate various embodiments of a temporary suture anchor knot that can be released and easily removed from the cortical hole according to the teachings of the present disclosure.

FIGS. 13 and 14 depict two embodiments of a beanbag or positioning spacer for use with the temporary suture anchor according to the teachings of the present disclosure.

FIG. 15 illustrates a modified embodiment of a navigational (e.g., optical, LIDAR, infrared, etc. technologies) tracker according to the teachings of the present disclosure.

FIG. 16 depicts an embodiment of a platform for use with the temporary suture anchor and navigational trackers according to the teachings of the present disclosure.

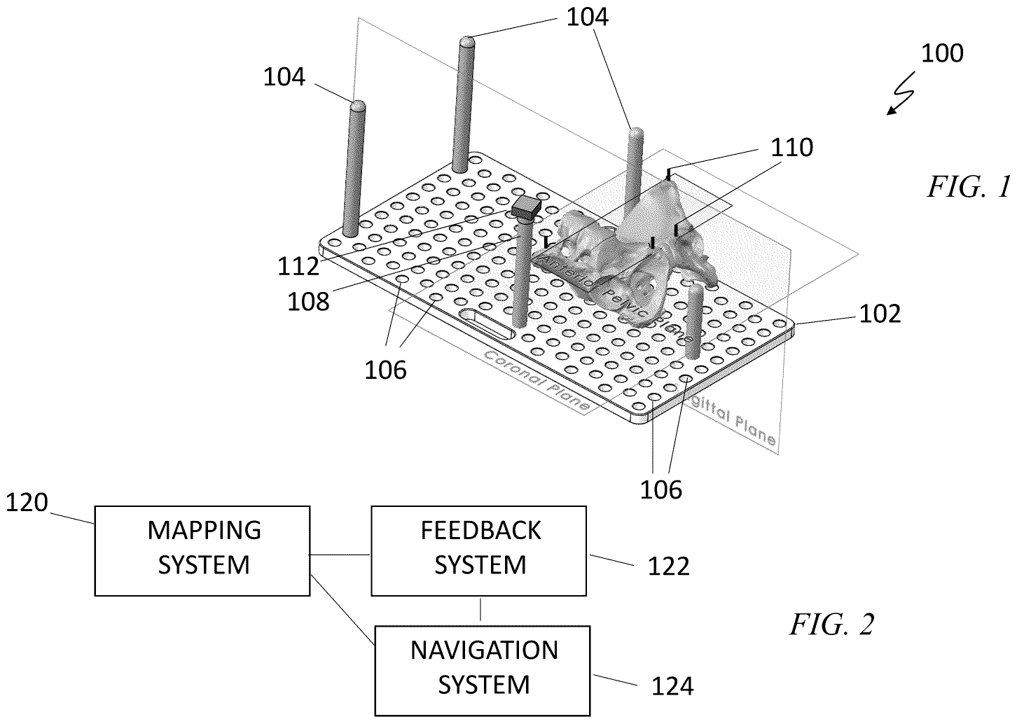

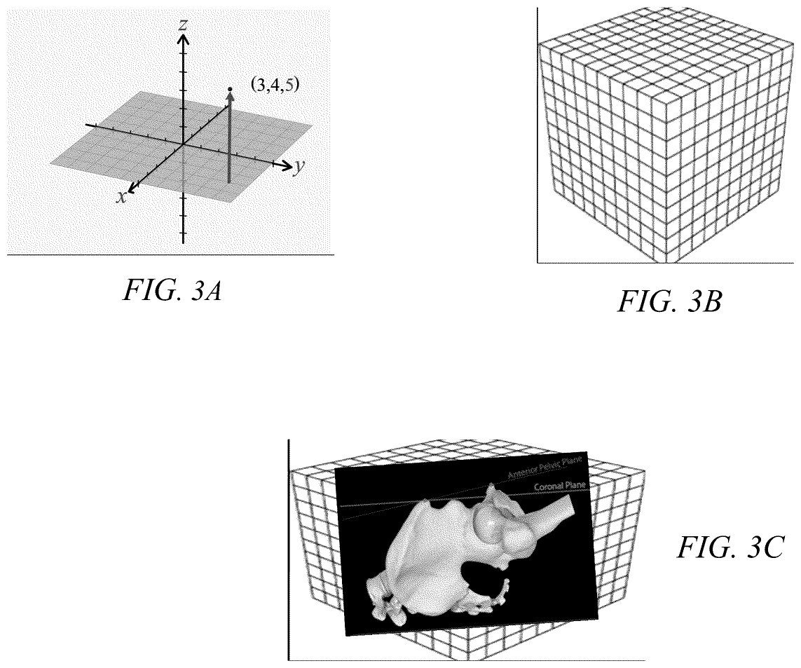

### DETAILED DESCRIPTION

The computerized system 100 described in the present disclosure includes a patient positioning device (e.g., an alignment board) 102; a dumb peg system that includes a plurality of dumb pegs 104 inserted in the plurality of holes 106 disposed in a matrix (or another pattern) on the alignment board 102 and disposed adjacent the patient's body to position and stabilize the patient; a smart peg system that includes one or more smart pegs 108 also inserted or secured to the alignment board 102; a tac system that includes a plurality of tacs (optical or other markers) 110 that are also secured to specific points on the patient's body or bone(s); and a mapping system 120 that detects the tacs 110 and smart pegs 108 as fixed reference points to determine the position and orientations of the patient's pelvis and proximal femur to create a spatial map of the patient's anatomy. These bone-fixed markers help to create a real-time coordinate system for the robotic system, so that the precise anatomy of the patient is known during the procedure.

A surgeon or another medical professional may use the system 100 described in this disclosure to perform a navigated or robotic surgery (example hip replacement surgery) on a patient. A spatial map is may be made of the patient's anatomy that will be operated on using CT (Computed Tomography) scan, MRI (Magnetic Resonance Imaging), or X-Rays. Points in the spatial map may be referenced by a three-coordinate plane system including: axial, coronal plane and a sagittal plane. To sync the patient's real time position on the operating table to the spatial map is made by placing the patient on the positioning device 102 (e.g., an alignment board with a plurality of holes 106 arranged in a matrix). The dimensions and shape of the alignment board 102 are tailored to fit securely on the operating table and provide ample coverage of the area around the patient's desired anatomy of interest (e.g., hip joint). The alignment board 102 is characterized by a grid of uniformly spaced holes 106 covering its surface. These holes 106 are designed to accommodate dumb and smart pegs 104 and 108 (i.e., positioning markers), aiding in accurately mapping the patient's anatomy. There are two types of pegs, including smart pegs 108 and dumb pegs 104. Once a smart peg 108 is inserted into a hole 106 on the alignment board 102, its location may be manually registered or automatically recognized (registered) by the mapping system by activating an electronic feedback system or by other means. The peg system establishes a series of boundaries that will define the area of interest. This gives the navigation system the proximal most, distal most, and height of the area of interest. The relevant anatomy will then be manually processed by a medical personnel to use a handheld probe tracked by the robotic/navigation system to touch specific anatomic landmarks and coordinate these locations with the existing tacs 110 and pegs 104 and 108. The electronic feedback system 122 may utilize a wireless communication system, such as radio-frequency identification, LIDAR, radar, infrared, electromagnetic tracking, pressure sensor, capacitive sensing, optical tracking system, or other suitable feedback systems to identify the location of the smart peg(s) 108 and tacs. For example, an electromagnetic tracking sensor 112 can be embedded in the smart peg 108, and the alignment board 102 can detect the smart peg's position in three-dimensional space through its electromagnetic tracking system. An electromagnetic field emitter creates a controlled electromagnetic field within the alignment board area. When the smart peg 108 is moved within the alignment board area, the electromagnetic tracking sensor 112 can detect changes in the electromagnetic signals. These changes vary depending on the electromagnetic tracking sensor's location relative to the emitter. The electromagnetic tracking system processes these signal variations to determine the exact location and orientation of the electromagnetic sensors 112 in real-time.

The dumb peg system includes at least one dumb peg 104. The dumb pegs 104 are also inserted into the holes 106 of the alignment board 102 and is used to position and stabilize the patient on the alignment board. The dumb peg system is used to position and hold the patient and is not recognized by the robotic/navigation system 124.

The smart peg system includes at least one smart peg 108. Each smart peg 108 is equipped with sensors, cameras, and/or markers that are detectable by a mapping system. The sensors or markers of the smart peg 108 can be electromagnetic sensors, radio-frequency identification tags, optical markers, ultrasonic markers, or other sensor or marks that enable cameras or scanners to detect or track their positions in real-time. The smart pegs 108 are inserted into the holes 106 of the alignment board 102. The smart pegs 108 are placed adjacent to specific anatomical landmarks around the patient's hip joint to stabilize the patient and aid in calculation of specific anatomical landmarks and thereby calculate pelvic planes and femoral reference points. The mapping system 120 determines and records the position of these anatomic landmarks relative to the smart pegs, effectively registering key points of the patient's hip anatomy. The mapping system 120 collects spatial data from the sensors or markers 112 on the smart pegs 108. The data is then used to generate a three-dimensional grid around the patient's anatomy of interest (e.g. pelvis and hip joint), a digital series of blocks of 1 mm³ or less in the X, Y, Z dimensions on a Cartesian graph. The real-time position and orientation of the patient's anatomical area of interest (e.g., pelvis and proximal femur) is captured by the sensors or markers and is recorded by the computer by adjusting the calibration. FIGS. 2A-2C illustrate the coronal, axial and the sagittal planes that define the patient's anatomical area of interest. By providing a clear visual landmarks and boundaries around the patient's anatomy, the smart pegs 108 help in minimize errors and improve surgical outcomes. The length of the smart pegs 108 and the relative position of the sensor 112 on the pegs are known to the mapping system.

The tac system used with the mapping system includes at least one tac 110. Each tac may be equipped with sensors or markers that interact with the computer-assisted system to provide a real-time navigation or positioning aid. The tacs 110 are affixed or secured to specific known points on the patient's anatomy. For example, the tacs 110 may be secured to the Anterior Superior Iliac Spine (ASIS) and/or Pubic tubercles on the patient's pelvis bone. Once the ASIS and pubic tubercles are referenced in the computer system, the anterior pelvic plane may be referenced and calculated. This anterior pelvic plane shows the pelvis position in reference to the patient's spine and this whole construct in relation to the peg system referenced above. From the anterior pelvic plane the patient's coronal, sagittal and axial planes maybe calculated with the assistance of the smart pegs referenced above. Unlike conventional pins that are screwed unicortically or bicortically into the patient's bones, the tacs 110 used herein have a much smaller diameter and are unicortical thus less invasive. In a preferred embodiment, the navigational trackers are affixed to the patient's bone using a temporary suture anchor. Hereafter, reference to the tacs 110 refers to the combined anchor point to bone, passage of material through the soft tissue with a reference tracker (optical vs other) that may be visible from the skin surface or referenced by the navigational system in some other way. The temporary suture anchoring device of the present disclosure are described in more detail below

The footprint of the tacs 110 varies in diameter but likely will be less than 4 mm in diameter and thereby lessens the adverse impact on the integrity of the patient's bones. The tacs 110 are trackable by the mapping system 120 by determining the relative positions of the tacs 110 in relation to the locations of the smart pegs 108. The mapping system 120 may interpret the positions of the tacs 110 in a generic computer model of a patient's hip geometry or this maybe overlaid onto a spatial map of the patient's actual anatomy if patient images are uploaded onto the navigation system (e.g., preoperative or intraoperative CT scans). A software module of the mapping system 120 defines the patient's anterior pelvic plane by locating ASIS tacs and referencing the pubic tubercles. Once these four points are referenced, the anterior pelvic plane maybe calculated and then the coronal plane, axial, and sagittal plane may be calculated from the navigation system 124.

As described above, the coronal plane is a vertical plane diving the body into anterior and posterior sections. The sagittal plane divides the body into left and right halves. The axial plane is a horizontal plane that separates the body into top and bottom halves. All these planes are 90 degrees from each other, so once one plane is identified, the others may be calculated. The anterior pelvic plane is defined by referencing the left and right ASIS and pubic tubercles. By identifying the anterior pelvic plane, the coronal plane maybe calculated, and from the coronal plane the other two planes can be located by spatial calculations. Any displacement of the patient's pelvis (movement of the anterior pelvic plane) is tracked by the position of the tacs 110 when referenced to the stationary and nonmobile smart pegs 108.

A femoral tac is fixed on the patient's proximal femur, lateral to the intertrochanteric line a medial and distal to the greater trochanter. The femoral tac is fixed to the femur in a firm and fully engaged position. The physician registers the femoral tac in relation to the pelvis tacs and smart pegs. This allows for calculation of the native femoral length and offset as well as native hip center of rotation which can either be done prior to the femoral osteotomy whereby the surgeon pivots the femur, typically in arcs or circles consistent with its natural arcs of movement, or after femoral head resection whereby a trial femoral ball is placed into the native acetabulum and this instrument is rotated. The movements of the femoral tac are tracked by the mapping system and interpreted by the computer to calculate the native center of rotation. After finding the native center of rotation, the physician resects the femur. The position of the femoral tac essentially defines a position on the native femoral length and offset prior to proximal femoral resection. This will give a “native X, Y, Z coordinate” that will be reassessed near the conclusion of the procedure. Near the conclusion of the procedure, during the trialing process, the physician is able to test various trial implants to determine the best size and fit based on restoring native anatomy and function based on pre replacement measurements. The femoral tac is registered again, and a new center of rotation, leg length and offset are calculated. (post procedure X, Y, Z coordinates of the hip center of rotation and post procedure femoral tac position) The mapping system again inputs the position of the femoral tac and calculates the change in leg length and offset as compared with those initialized previously by using a delta femoral tac position. (Difference between pre procedure femoral tac position vs post procedure femoral tac position when comparing X, Y, Z coordinates)

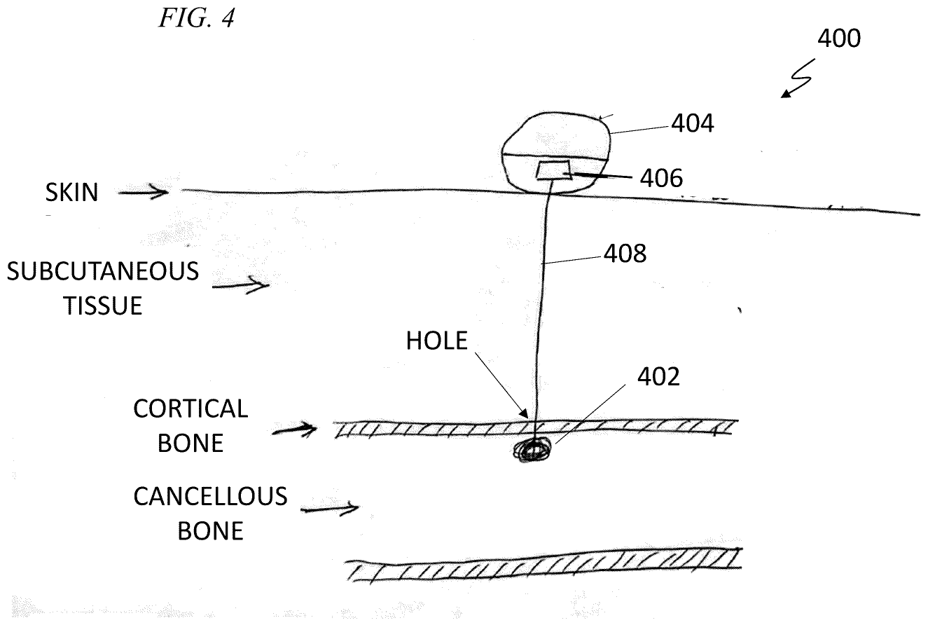

Turning to FIGS. 4-16, specific embodiments of temporary suture anchors used to temporarily secure the tacs or trackers to the patient's anatomy are shown and described below. The term “tracker,” “optical tracker,” or “navigational tracker” are also used to refer to the devices affixed to the patient's anatomy to enable mapping of the patient's anatomy of interest from the skin surface and provide guidance for robotic navigation, for example. As referenced above, conventional trackers have been affixed to the patient's pelvic bone using invasive bone screws or threaded pins. The innovative temporary suture device shown in FIGS. 4-16 and described herein is used to secure a navigational tracker to the patient's anatomy, such as an optical tracker that allows for camera recognition of the optical tracker without the need for screw or pin fixation. Alternatively, automatically trackable technologies other than optical techniques may be used.

As shown in FIG. 4, an embodiment of the innovative temporary suture device 400 to secure a tracker includes a suture anchor 402 is used to provide temporary fixation of the tracker 404 to the patient's anatomy. The tracker 404 may be a visual marker that robotics vision is able to “see” accurately. The tracker 404 is securely tethered to a suture 406 at one end. The tracker 404 may include a hollow shell or container that contains a release suture 406. The other end of the suture 406 is the temporary suture anchor 402, preferably under 4 mm in diameter, that is inserted unicortically into bone using a hollow inserter tool or inserting guide with a sharp end (not explicitly shown). The inserter is then removed and the anchor 402 is deployed to temporarily but securely hold the optical tracker 404 to the patient.

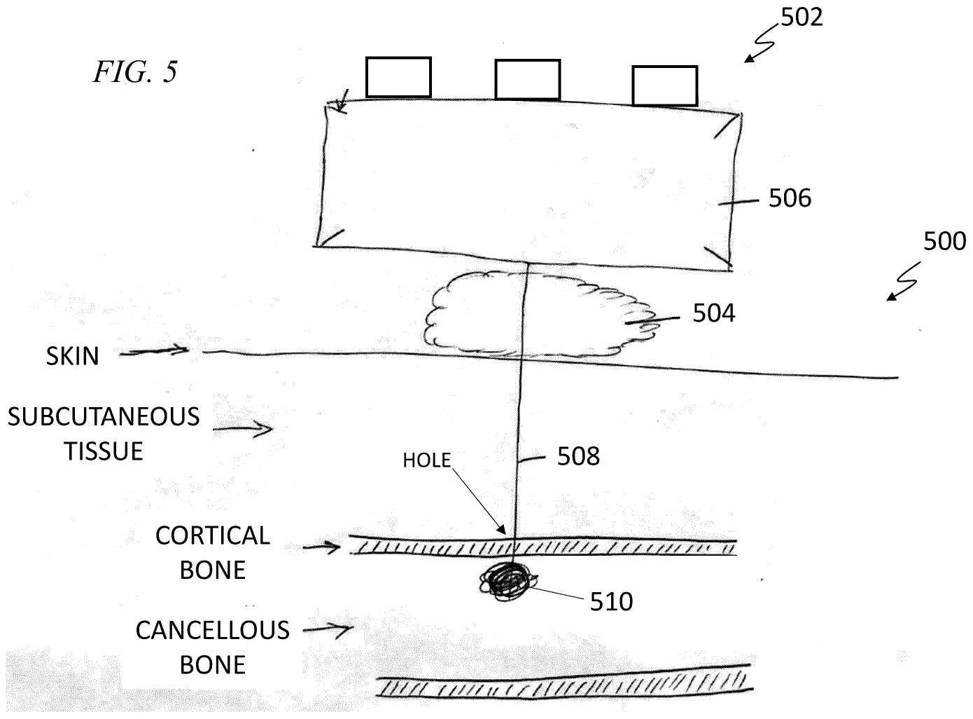

To deploy the anchor 404 one or more interconnected sutures 408 are pulled taut to tighten a short leash (shown in FIG. 4) or a loop (or series of loops) underneath the cortical bone and within the cancellous bone area. Various embodiments of the suture loop are described below and show in FIGS. 5-16. By tightening this deeper loop (or series of loops) the implanted end of the sutures expands in size and create a “suture ball” or “suture knot” that has a larger diameter than the insertion hole, providing an anchor for the optical tracker 404 that will be placed adjacent to the patient's skin. The inserting guide tool is then removed and the excess length of the sutures are pulled with the optical tracker 404 attached. The sutures are tightened until the optical tracker 404 is resting against the patient's skin, as shown in FIG. 4. At the conclusion of the surgery the optical tracker 404 can be opened to reveal the release suture 406. Using the release suture 406, the suture anchor 402 is unknotted and the suture 408 may be removed completely from the patient's anatomy without retaining any anchor component.

Referring to FIG. 5 for an alternate embodiment of a suture anchor device 500 for securing one or more trackers 502. In situations where a large tracker 502 is needed, a combination of a beanbag 504 and a platform 506 may be assembled and used to support the tracker 502. The beanbag (filled with small pellets) 504 can be placed on the patient and allow its shape and contour to closely conform with the patient's anatomy and support the platform 506 that holds the tracker 502. The suture that passes through the soft tissues becomes a “short leash” 508 connected to the optical tracker 502 that rests against the patient's skin so that bony movement will be visible from the skin surface. At the other end of the suture 508 is the suture anchor 510 that is inserted into a hole in the bone. At the end of the surgery, when the navigation trackers 502 are no longer needed, the sutures 502 are released from the optical tracker 502. The sutures that were used to form the loops (i.e., anchor) within the unicortical window or hole are then pulled. Once the “looping sutures” are removed the remaining suture will collapse in size and will be removed thereby only leaving a unicortical window less than 4 mm in diameter. In case any of these sutures break or are unable to be removed the suture may be made with dissolvable sutures.

FIG. 4 shows a small hole that has been punched unicortically into the patient's cortical bone and the insertion and deployment of the suture anchor. The remaining suture is then pulled tight so that the superficial construct rests snugly against the patient's skin. In cases where multiple anchors may be needed or in cases where a larger navigation array needs to be fixed, the beanbag 504 and platform 506 may be used (FIG. 5). The beanbag 504 allows load distribution of the supported trackers 502 on the platform 506 so as not to compromise the skin and will also allow for accommodating the variability of different body contours. The beanbag 504 and platform 506 hold the sutures taut and in place, not allowing them to release prematurely. The platform 506 may have holders to affix the navigation/robotic trackers 502. For example, the trackers 502 may be held to the platform 506 via magnets, clips, or some other fastening device. These trackers 502 may be circular, rectangular, or irregularly shaped and maybe uniform in appearance. At the conclusion of the surgery, when the surgeon no longer requires robotics or navigation, the sutures maybe released thereby unknotting the suture anchor 510 and allowing the entire construct to disengage and be removed completely.

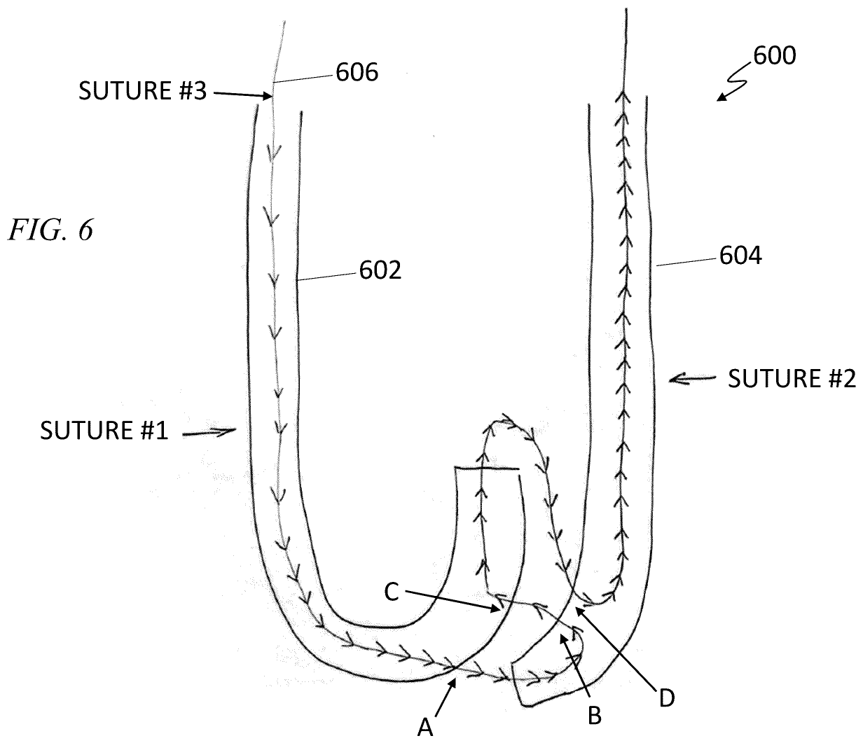

In FIG. 6, an embodiment of the suture anchor structure 600 is shown. Suture 602 (suture #1) and suture 604 (suture #2) are both hollow and each forms an elongated passageway. Suture #1 and #2 allow for a third suture (suture #3) 606 to pass inside and through the walls of the hollow sutures #1 and #2. In this anchor design suture #3 606 passes through suture #1 and exits through its wall near the base of the bend (at point A). Suture #3 then enters suture #2 at one end and exits a few millimeters from the entrance (at point B) and then exits again to reenter suture #1 (at point C) near the end of suture #1 to exit the end of suture #1. Suture #3 then reenters suture #2 (at point D) and proceeds out of the end of suture #2. Suture #3 is used to tether to the trackers at or elevated above the patient's body.

As shown in FIG. 6, when suture #3 is pulled tight it will cause the lower end of sutures #1 & #2 to knot and ball up around each other. This will produce a knot diameter that will be much larger than the small cortical hole made by the inserter tool. At the termination of the orthopedic procedure, suture #3 will be pulled out from the skin edge superficially. This will allow for the lumen of sutures #1 and #2 to collapse and disengage from each other. Suture #1 and suture #2 may then be pulled out from the skin edge thereby leaving no suture anchor remnants within the patient.

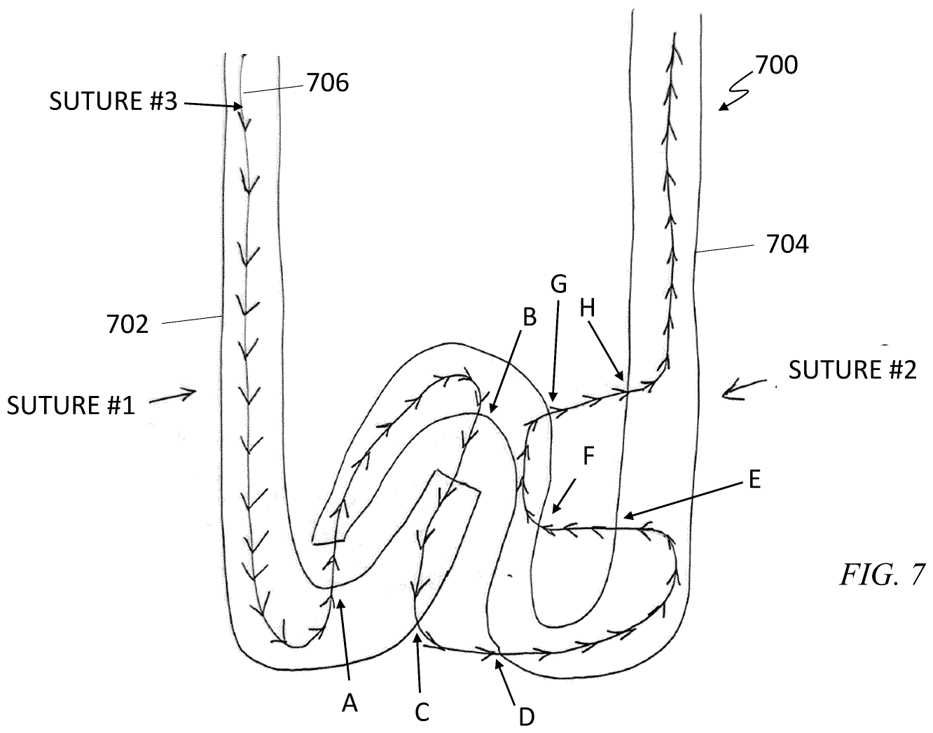

FIG. 7 shows another embodiment of the suture anchor structure 700. Suture 702 (suture #1) and suture 704 (suture #2) are both hollow and each forms an elongated passageway. Suture #1 and #2 allow for a third suture (suture #3) 706 to pass inside and through the walls of the hollow sutures #1 and #2. In this anchor design suture #1 at has a hook-like structure at its end and suture #2 has a “lazy S”-shaped design at its end. Suture #3 passes through and exits both sutures #1 and #2 repeatedly. Suture #3 706 passes through suture #1 and exits through its wall near the base of the bend (at point A). Suture #3 then enters suture #2 at one end and exits suture #2 a few millimeters from the entrance (at point B) and then enters one end of suture #1 and exits again to reenter suture #1 from its end, only to exit again (at point C) near the end of suture #1. Suture #3 then reenters suture #2 (at point D), exits (at point E), reenters suture #1 (at point F), exits (at point G), and reenters suture #2 (at point H), and proceeds out of the end of suture #2. When suture #3 is pulled tight (secured at one end and pulled from the second end), the action of suture #3 pulls the ends of sutures #1 and #2 together, and because suture #3 is essentially interwoven between sutures #1 and #2, their ends form a “suture knot” of a diameter that is much larger than the small cortical hole by the inserter. At the termination of the orthopedic procedure, suture #3 is pulled out from the skin edge superficially. This allows for the lumen of sutures #1 and #2 to collapse and their ends to disengage from each other. Sutures #1 and #2 may then be pulled out from the skin edge thereby leaving no suture anchor remnants within the patient.

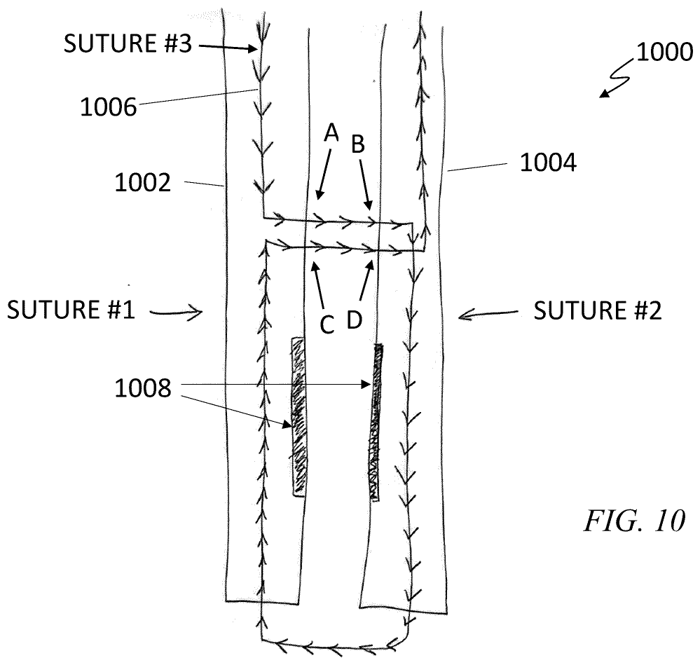

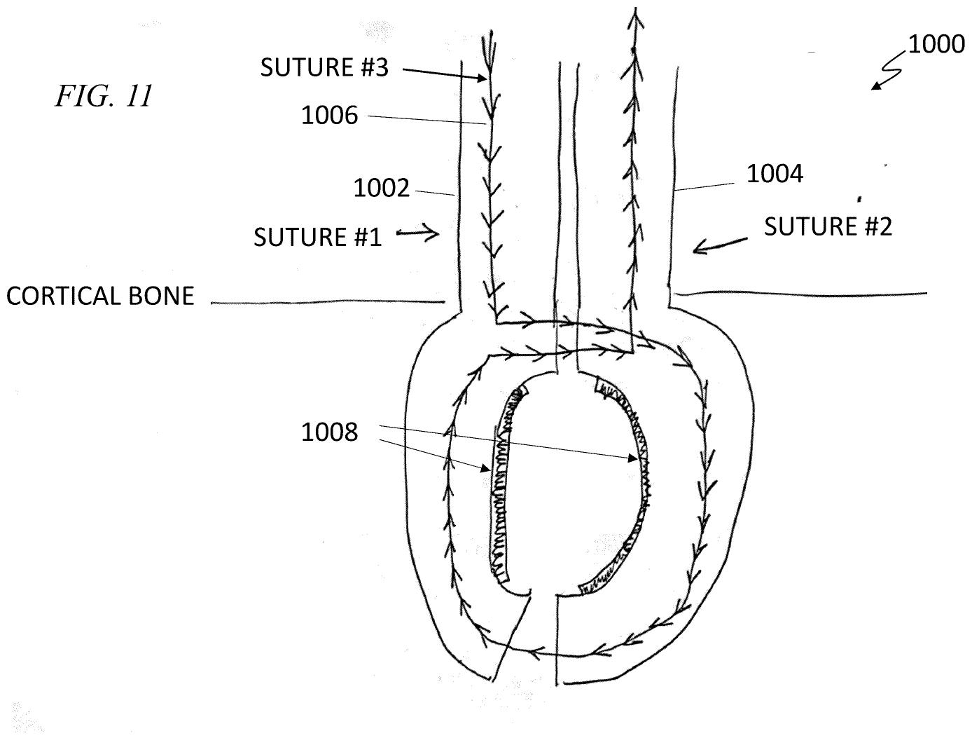

FIGS. 10 and 11 show yet another embodiment of the suture anchor 1000. Suture #1 1002 and suture #2 10004 are both hollow and their passageways allow for a third suture (suture #3 1006) to pass inside and through their walls (at points A-D) in a “figure 8” or H-pattern. In this design there is a portion removed from the walls of sutures #1 and #2, forming openings 1008. As shown in FIG. 11, when suture #3 is pulled tight (secured at one end and pulled from the second end) in this configuration, sutures #1 and #2 form a knot and ball up folding in at the areas about the openings 1008, thereby increasing the overall size of the knot to be larger than the diameter of the cortical hole and enabling the knot to be securely anchored inside the hole. At the termination of the procedure, suture #3 is pulled out from the skin edge superficially. This allows the lumen of sutures #1 and #2 to collapse and disengage from each other. Suture #1 and suture #2 may then be pulled out from the skin edge thereby leaving no suture anchor remnants within the patient.

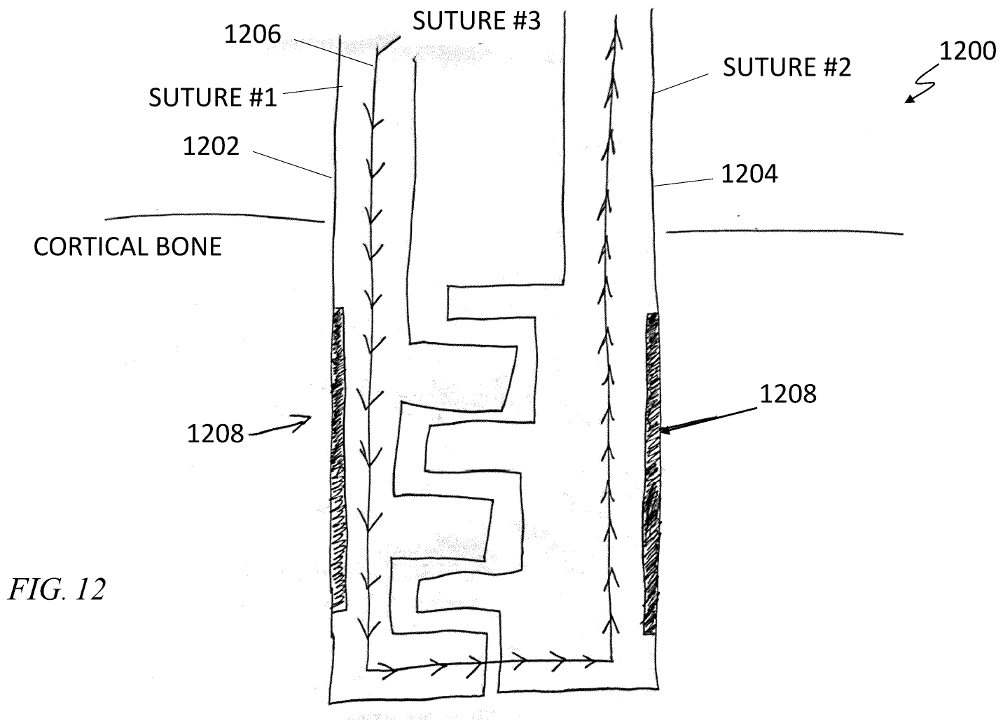

FIG. 12 shows another embodiment of the suture anchor 1200 that has a “zipper”-like interconnection between suture #1 1202 and suture #2 1204. Both of these sutures are hollow and allow for suture #3 1206 to pass through their passageways. Wall portions of sutures #1 and #2 are removed to form openings 1208. This allows suture #3 to be pulled and tightened (secured at one end and pulled from the second end) to cause a collapse of the walls of the sutures #1 and #2 around the openings 1208, increasing the size of the resultant suture knot anchor to be larger than the cortical hole. At the termination of the procedure, suture #3 is pulled out from the passageways of skin edge superficially, and allows the lumen of sutures #1 and #2 to collapse and disengage from each other. Suture #1 and suture #2 may then be pulled out from the skin edge sequentially thereby leaving no suture anchor remnants within the patient.

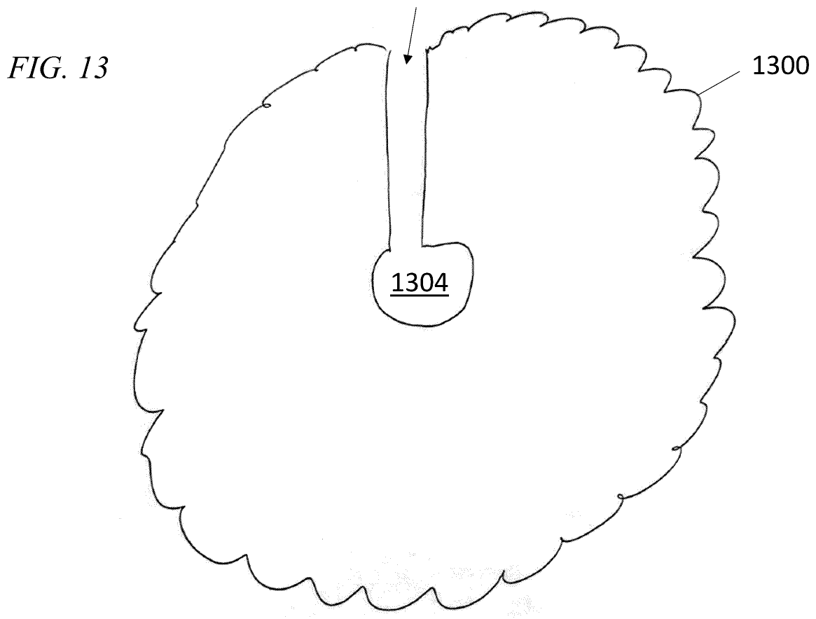

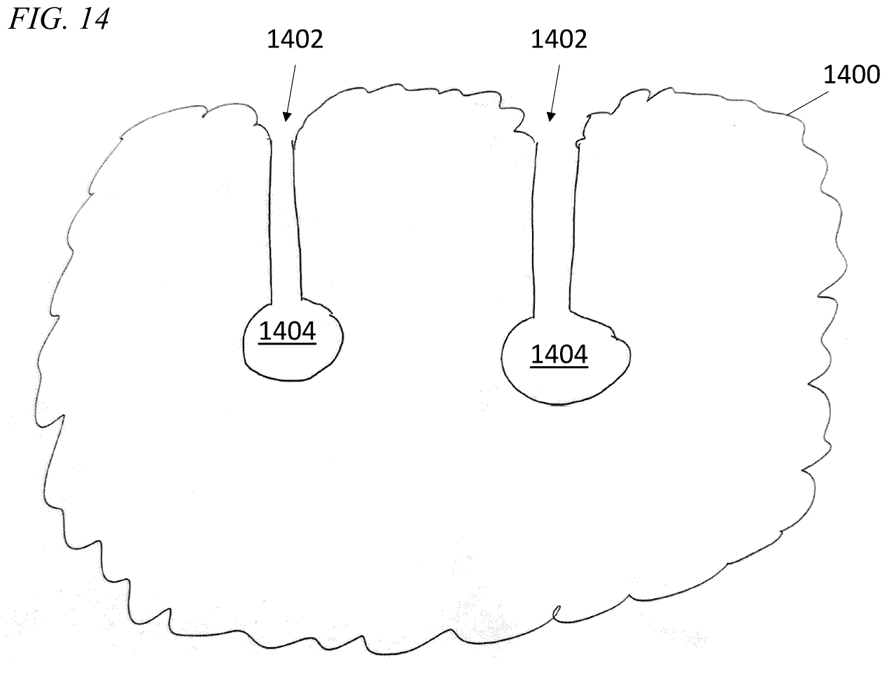

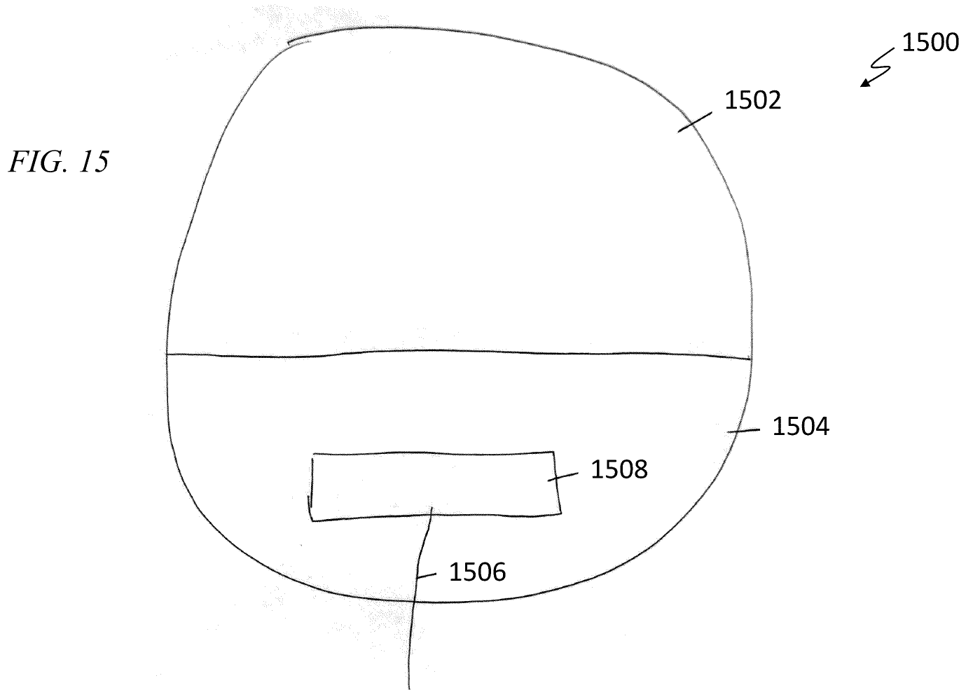

FIG. 13 is a rough sketch of one embodiment of the beanbag 1300 that may be used to help distribute the load of a heavy tracker system or the platform shown in FIG. 5 described above. The outer mesh layer of the beanbag 1300 encloses a collection of small beads or pellets. These beads can be loosely arranged so that when the beanbag 1300 is compressed between the skin and the platform the beanbag will contour to the patient's body habitus. The beanbag 1300 has a generally cut doughnut or cut torus configuration (i.e., a torus with a radial cut). The radial slit 1302 in the beanbag 1300 allows the “short leash” suture portion of the anchor to pass to the center 1304 of the beanbag without compromising the integrity of the beanbag. FIG. 14 shows another embodiment of the beanbag 1400 that has two openings 1404 and two slits 1402 to accommodate two suture anchors. It should be noted that the size, shape, and number of openings and slits can be variable to adapt the needs of the surgeon and the surgery to be performed. The bean bags may be disposable or are constructed of materials that can be economically sanitized and sterilized.

FIG. 15 shows an embodiment of a modified embodiment of an optical tracker 1500. The optical tracker 1500 includes a hollow shell and has a top or lid portion 1502 that can be disengaged from a bottom portion 1504. Top portion 1502 may be hinged to the bottom portion 1504. Inside of bottom portion 1504 is a tensioning device that will allow the ends of the “suture leash” 1506 (remainder of the suture that is visible from the skin edge) to be tightened and held taut by a locking mechanism 1508. At the termination of the procedure when the optical tracker 1500 is no longer needed then the tracker may be opened by opening lid portion 1502 and thereby revealing the locking mechanism 1508. The locking mechanism 1508 is unlocked to allow the suture anchor to be undone. The locking mechanism 1508 may be any known or existing device (also any later developed device) that enables quick shortening of the suture leash and secure the ends of the sutures to keep the suture anchor in place in the cortical hole during the surgical procedure.

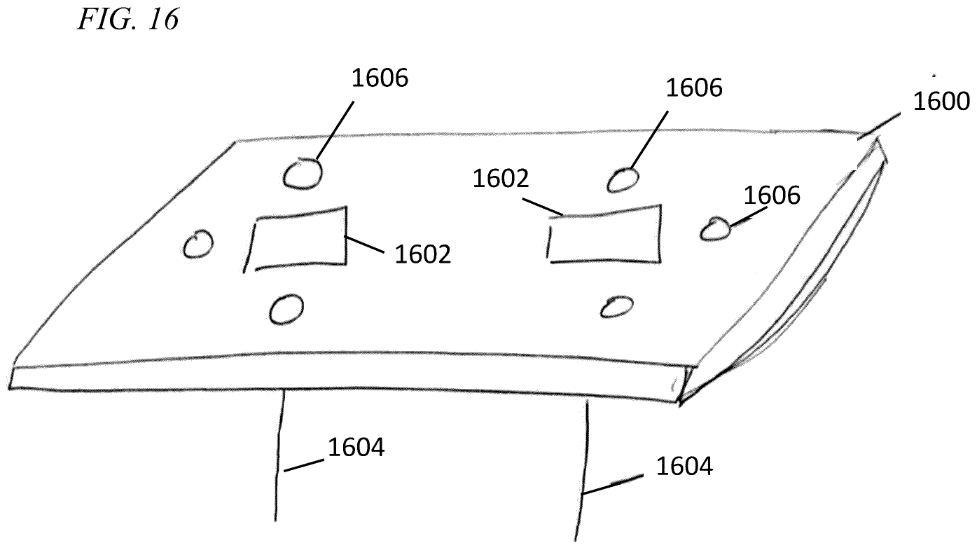

FIG. 16 shows an embodiment of the platform that includes a generally planar plank 1600 and locking mechanisms 1602 that can be used to tighten and secure the sutures 1604 that extend from the patient's skin. The platform 1600 may include a number of magnets 1606 that are used to affix the navigation trackers to the platform. The magnets 1606 are arranged in a way to align the navigation trackers in the same orientation each time. At the termination of the surgical procedure, when navigation is no longer needed, the locking mechanism 1602 is undone to allow the suture anchor to unravel and be disassembled.

In summary, the method of using the temporary suture anchor described herein includes the steps of: 1) forming a cortical hole with a small diameter awl, punch, drill, or another suitable tool; 2) insertion of the undeployed anchor using a hollow inserter tool; 3) deployment of the anchor (such as tightening the suture to form a knot ball inside the cortical hole); 4) fixation of the superficial sutures to the tracker; 4a) use a beanbag and platform if needed to deploy and stabilize the tracker in place. At the termination of the surgical procedure, 5) unlocking the sutures from the tracker or platform; 6) release of the anchor deployment; and 7) removal of all anchor segments.

Although the use of suture anchors exists in orthopedics currently, the primary purpose of the current suture anchors is to affix damaged tendons, ligaments, or muscles to intact bone, which is meant to be a permanent fixation. In these conventional processes, the anchors are designed to remain permanently in place and are eventually incorporated into the body. The novel suture anchor device described herein differs from those previous anchors in that it is designed to be a temporary fixation method for the purpose of holding navigation or robotic trackers in place during the surgical procedure. Since this will be a temporary fixation, the sutures involved are intended to be removed. The “suture knot” that is created for temporarily affixing the tracker is to be “released and unknotted” at the end of the procedure thereby leaving nothing behind within the patient. In case there is a fraying or damage to the suture anchor upon removal, the suture anchor material is preferably made of dissolvable materials. The dissolvable suture anchor material also differs from the current suture anchors available today that are intended for permanent implantation and eventual incorporation into the body.

It should be noted that the 3D pinless navigation/robotic system and method can be used for hip, shoulder, knee, elbow, wrist, or other body part replacements or for navigated instrumentation into the spine. The method and system can be adapted to various body geometries and sizes, and could even be adapted, with proper modifications for veterinary surgery. The geometries of the various tools, pegs, tacs, and markers can be varied or modified to accommodate different tracking approaches and can be adapted for periacetabular surgery, open reduction internal fixation surgery or other surgical procedures.

It should be noted that the smart pegs may be trackable by the mapping system using a variety of technologies, such as optical and wireless sensors disposed atop of the pegs. Moreover, the positioning device may be equipped with sensors that can detect the presence of the smart pegs in its grid of holes, and to deduce their location using this knowledge.

The features of the present disclosure which are believed to be novel are set forth below with particularity in the appended claims. However, modification, variations, and changes to the exemplary embodiments described above will be apparent to those skilled in the art, and the disclosure described herein thus encompasses such modification, variations, and changes and are not limited to the specific embodiments described herein.

What is claimed is:

1. A method of temporarily affixing a robotic navigational marker to a patient's anatomy for an orthopedic procedure, comprising:
forming a small diameter superficial hole in a bone;
inserting a knot end of a suture anchor device into the superficial hole, the suture anchor further having a leash end coupled to the knot end by a leash segment;
pulling the leash end and producing an enlarged knot end that expands within the superficial hole in the bone;
securing and tightening the leash end to the navigational marker; and
placing the navigational marker against the patient's skin to reference a deeper anatomy.

2. The method of claim 1, further comprising using a beanbag to support the navigation marker against the patient's skin.

3. The method of claim 1, further comprising releasing the leash end and causing the knot end to loosen and be withdrawn from the superficial hole in the bone.

4. The method of claim 1, further comprising resting the navigational marker on a beanbag adapted to conform to the patient's anatomical contours.

5. The method of claim 1, further comprising securing the navigational marker to a platform and resting the platform on a beanbag adapted to conform to the patient's anatomical contours.

6. A suture anchor for temporarily affixing a navigation marker to a patient's anatomy for an orthopedic procedure, comprising:
a first suture having a hollow passageway connecting a knot end and a leash end;
a second suture having a hollow passageway connecting a knot end and a leash end;
a third suture having two leash ends and passing through the hollow passageways of the first and second sutures and interconnecting the knot ends thereof;
wherein pulling the two leash ends of the third suture causes the knot ends of the first and second sutures to interlock and expand into an enlarged knot; and
wherein the enlarge knot is adapted to engage a small diameter superficial hole in a patient's bone.

7. The suture anchor of claim 4, wherein the knot ends of the first and second sutures have interlocking hook formations.

8. The suture anchor of claim 4, wherein the knot ends of the first and second sutures have openings formed in their outer walls that causes a collapse of the knot ends of the first and second sutures and formation of the enlarge knot when the third suture is tightened.

9. The suture anchor of claim 4, wherein the knot ends of the first and second sutures have interlocking levers.

10. The suture anchor of claim 4, wherein the leash ends of the third suture is used to secure a navigational tracker, and a beanbag is used to support the navigational tracker against a patient's anatomy.

11. The suture anchor of claim 8, wherein the beanbag has a torus shape with a radial cut.

12. The suture anchor of claim 8, wherein the beanbag has at least one center opening with at least one through slit.

13. A 3D pinless navigation robotics method, comprising the steps of:
placing a patient on a positioning device that tracks patient alignment;
attaching at least one dumb peg to the positioning device to position and stabilize the patient;
attaching at least one trackable smart peg to the positioning device;
fixing at least one trackable tac on at least one known location on at least one body part of
the patient using a temporary suture anchor affixed to a small diameter superficial hole in the patient's bone; and
mapping the locations of the at least one trackable peg and the at least one trackable tac and creating a spatial map of the patient's at least one body part relative to the at least one trackable peg and tac.

14. The method of claim 11, further comprising releasing the temporary suture anchor from the patient's bone and removing the suture anchor and at least one trackable tac.

15. The method of claim 11, wherein fixing a trackable tac comprises:
forming a small diameter superficial hole in the patient's bone;
inserting a knot end of a suture anchor device into the superficial hole, the suture anchor further having a leash end coupled to the knot end by a leash segment;
pulling the leash end and producing an enlarged knot end that expands within the superficial hole in the bone;
securing and tightening the leash end to the trackable tac; and
placing the trackable tac to reference a deeper anatomy.

16. The method of claim 13, further comprising using a beanbag to support the trackable tac against the patient's skin.

17. The method of claim 13, further comprising releasing the leash end and causing the knot end to loosen and be withdrawn from the superficial hole in the bone.

18. The method of claim 13, further comprising securing the trackable tac to a platform and resting the platform on a beanbag adapted to conform to the patient's anatomical contours.
